(19) United States
(12) Patent Application Publication
(10) Pub. No.: US 2004/0266739 A1
(43) Pub. Date: Dec. 30, 2004

(54) Composition comprising minocycline as an effective component for prevention and treatment of dementia, and learning and memory impairments

(76) Inventors: Yoo-Hun Suh, Seoul (KR); Hye-Sun Kim, Seoul (KR); Cheol-Hyoung Park, Seoul (KR); Eun-Mee Kim, Seoul (KR); Ki-Young Shin, Kwangju (KR)

Correspondence Address: GREENBLUM & BERNSTEIN, P.L.C., 1950 ROLAND CLARKE PLACE, RESTON, VA, 20191, US

(21) Appl. No.: 10/657,143

(22) Filed: Sep. 9, 2003

(30) Foreign Application Priority Data
Sep. 10, 2002 (KR) 10-2002-0054650

(51) Int. Cl. A61K 31/65

(52) U.S. Cl. 514/152

(57) ABSTRACT

The present invention relates to a composition for preventing and treating dementia and memory impairment, which contains minocycline as active ingredient. The composition of the present invention has an effect of inhibiting brain cell death and memory impairment, which are induced by amyloid beta-protein and C-terminal protein. Thus, the composition of the present invention is useful for the prevention and treatment of various dementias, including Alzheimer's disease, and the impairment of learning and memory and cognitive function.

### BACKGROUND OF THE INVENTION

1. Field of the Invention

The present invention relates to a composition for preventing and treating dementia and memory impairment, which contains minocycline as an active ingredient.

2. Background of the Related Art

Dementia is a degenerative disease characterized by the appearance of general brain malfunctions, such as brain cell death and memory impairment, etc., and is estimated to affect about 10% of persons sixty-five years of age or older and about 50% or more of persons eighty years of age or older. With a recent increase in old age population, it is expected that the development rate of dementia will be more rapidly increased. Thus, dementia is becoming serious social and economical problems.

Dementia is caused by various diseases and divided into Alzheimer's disease, vascular dementia, and other dementias caused by alcoholism, trauma and a sequela of Parkinson's disease. Since more than half of dementia patients show Alzheimer's disease, studies on dementia are centering on Alzheimer's disease.

Although the mechanism of Alzheimer's disease was not clearly established, Alzheimer's disease is known as being closely connected with the reduction of function of acetylcholine in the central nervous system, the deposition of amyloid beta (Aβ) protein-based senile plaque in brain cells, or the appearance of extracellular hyperphosphrylated tau protein-based neurofibrillary tangle, etc.

In the prior art, there were many research results showing the correlation between memory impairment and the reduction of function of the cholinergic nervous system among such causes. Thus, there have been continued efforts to supplement and improve the cholinergic nervous system, thereby treating dementia.

Accordingly, existing agents for treating dementia are mostly substances, such as an acetylcholinesterase inhibitor capable of inhibiting the activity of acetylcholine esterase in the brain, or an acetylcholine precursor and receptor agonist capable of inhibiting the concentration of acetylcholine in the brain at a given level.

Examples of the acetylcholinesterase inhibitor include tacrine, aricept and galantamine, which were approved by FDA and are in the market in many countries including Korea. Also, the acetylcholine precursor includes lecitin, and the acetylcholine receptor agonist includes RS-86, nicotine and the like.

However, as dementia progresses, acetylcholinergic nerve cells and also glutamate nerve cells forming most of nerve cells are degenerated, and thus, the above-mentioned method centering on the increase of activity of acetylcholine achieves only temporary and weak effects in the prevention and treatment of dementia.

Furthermore, most drugs including tacrine still have many rooms for dispute on their use due to side effects, such as hepatic toxicity, emesis, nausea, diarrhea and the like.

Recently, there are reported research results indicating that C-terminal protein linked to the C-terminal region of amyloid precursor protein shows a stronger toxicity than amyloid beta-protein already known as a dementia-causing factor while acting as a main cause of dementia.

Considering this fact, drugs capable of inhibiting the production of such proteins and the effect of brain cell toxicity induced by such protein, for example, an inhibitor of toxic amyloid protein production, a normal metabolism promoter and an inhibitor of toxic amyloid protein deposition, are currently under development.

However, substances showing a positive effect have not yet been reported, and amyloid beta-protein immunotherapy which was clinically applied, is no longer used because of the side effect of inflammatory reaction in the central nervous system.

Accordingly, there is an urgent need for drugs capable of preventing and treating dementia and memory impairment without particular side effects.

Meanwhile, minocycline, a tetracycline-based antibiotic, shows excellent effect against a wide range of bacteria, virus and intestinal protozoa, and is known as a drug having verified safety without side effects even in long-term use for persons.

Recently, in animal models of Huntington's disease, amyotrophic lateral sclerosis and Parkinson's disease, neurodegenerative diseases, minocycline was recently proved to have another effect of inhibiting the expression and activity of caspase-1 and caspase-3 proteases promoting abnormal protein metabolism, and also inhibiting the activity of p38 MAPK contributing to apoptotic signal transduction to induce cell death, thereby blunting these diseases.

The present inventors have found that minocycline has an effect of preventing and treating various dementias, including Alzheimer's disease, and memory impairment, and as a result, perfected the present invention.

### SUMMARY OF THE INVENTION

Accordingly, an object of the present invention is to provide a composition for preventing and treating dementia and memory impairment, which contains minocycline as an active ingredient and shows inhibitory effect against brain cell death and memory impairment.

The present invention provides a composition for preventing and treating dementia, which contains minocycline as an active ingredient.

Also, the present invention provides a pharmaceutical composition for preventing and treating memory impairment, which contains minocycline as an active ingredient.

The composition of the present invention inhibits brain cell toxicity induced by amyloid beta-protein. Furthermore, the composition of the present invention inhibits brain cell toxicity induced by C-terminal protein. Thus, the composition of the present invention can inhibit brain cell death, a main symptom of dementia.

The composition of the present invention inhibits memory impairment induced by amyloid beta-protein. Furthermore, the composition of the present invention inhibits memory impairment induced by C-terminal protein. Thus, the composition of the present invention can inhibit memory impairment and thus learning impairment, main symptoms of dementia.

Since brain cell death and memory impairment are main symptoms occurring in Alzheimer's disease, the composition of the present invention is highly useful for the prevention and treatment of Alzheimer's disease among dementias.

In the composition of the present invention, minocycline is dissolved in physiological buffer solution to suitable concentration before use.

In addition to minocycline, the composition of the present invention may also contain an active ingredient having equal or similar functions to minocycline, and if necessary, other active ingredients.

Toxicity test results for the composition of the present invention showed that it had no particular side effects on the living body.

In addition to the active ingredients as described above, the composition of the present invention may contain pharmaceutically suitable, physiologically acceptable additives. Such additives include solubilizers, such as solvents, disintegrants, sweeteners, binders, coating agents, lubricants, and flavorings.

The pharmaceutical composition of the present invention may be formulated with one or more pharmaceutically acceptable carriers for administration, in addition to the active ingredients as described above.

Examples of the pharmaceutically acceptable carriers include saline, sterilized water, Ringer's solution, dextrose solution, maltodextrin solution, glycerol, ethanol, and a mixture thereof. If necessary, other conventional additives, such as antioxidants, buffer solutions and bacteriostatics, etc. may also be added. In addition, diluents, dispersants, surfactants, binders and lubricants may also be added so that the composition of the present invention can be formulated as formulations for injection, such as solution, suspension and emulsion, pills, capsules or tablets. Furthermore, the composition of the present invention can be preferably formulated according to diseases or components, by a method suitably used in the relevant field of the art, or a method described in Remington's Pharmaceutical Science (the latest edition), Mack Publishing Company, Easton Pa.

The pharmaceutical composition of the present invention can be formulated as granules, powders, coated tablets, tablets, capsules, suppositories, syrup, juice, suspension, emulsion, drops, injectable liquid, and sustained-release preparations of active ingredients, etc.

The pharmaceutical composition of the present invention can be administered in the conventional manner via the intravenous, intraarterial, intraabdominal, intramusclar, intrasternal, percutaneous, intranasal, inhalation, topical, rectal, oral, intraocular or intradermal route.

The dose of the pharmaceutical composition according to the present invention is preferably in the range of 100-200 mg/day for adults. This dose can vary depending on various factors, including the kind of diseases, the severity of diseases, the kind and content of active ingredients and other components contained in the composition, the kind of a formulation, and patient's age, weight, general health condition, sex and diet, and administration time, administration route, the secretion % of the composition, administration period, and the kind of drugs used in combination with the composition.

The pharmaceutical composition of the present invention can be used alone or in combination with a surgical operation, radiation therapy, hormone therapy, chemical therapy, or therapies using biological response modifiers.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIGS. 1a and 1b are drawings showing that a composition of the present invention can inhibit the effect of brain cell toxicity induced by extracellularly treated amyloid beta-protein;

FIGS. 2a and 2b show the results of MTT assay and LDH assay, which indicate that a composition of the present invention can inhibit the effect of brain cell toxicity induced by intracellularly produced amyloid beta-protein;

FIG. 3 shows the results of Cyt C assay, which indicate that a composition of the present invention can inhibit the effect of brain cell toxicity induced by intracellularly produced amyloid beta-protein;

FIGS. 4a and 4b show the results of MTT assay and LDH assay, which indicate that a composition of the present invention can inhibit the effect of brain cell toxicity induced by intracellularly produced C-terminal protein;

FIG. 5 shows the results of Cyt C assay, which indicate that a composition of the present invention can inhibit the effect of brain cell toxicity induced by intracellularly produced C-terminal protein;

FIG. 6 is a drawing showing that a composition of the present invention can recover the impairment of simple memory in dementia animal models induced by C-terminal protein;

FIG. 7 is a drawing showing that a composition of the present invention can recover the impairment of spatial memory in dementia animal models induced by C-terminal protein;

FIG. 8 is a drawing which shows, according to time periods, that a composition of the present invention can recover the impairment of memory and learning ability in dementia animal models induced by amyloid beta-protein; and

FIG. 9 is a drawing which shows, according to test zones, that a composition of the present invention can recover the impairment of spatial memory in dementia animal models induced by amyloid beta-protein.

### DETAILED DESCRIPTION OF THE PREFERRED EMBODIMENT

The present invention will hereinafter be described in further detail by examples. It should however be borne in mind that the present invention is not limited to or by the examples.

### EXAMPLE 1

### Effect of Inventive Composition on Brain Cell Death Induced by Extracellularly Treated Amyloid Beta-Protein

The effect of the composition of the present invention on brain cell death induced by extracellularly treated amyloid beta-protein was examined as follows.

1) Production of Inventive Composition

Minocycline was dissolved in PBS buffer (pH 7.4) to a concentration of 10 mM to produce the composition of the present invention.

2) Preparation of Amyloid Beta-Protein

As amyloid beta-protein to be treated extracellularly, an Aβ₁₋₄₂ protein manufactured by U.S. peptide inc. was purchased.

3) Cell Culturing

For use as neuronal models, P12 cells originated from rat pheochromocytoma were selected. The cells were cultured in DMEM medium containing 10% fetal bovine serum and 1% antibiotic, and two days before tests, were differentiated into nerve cells by low-serum (0.3%) medium containing 50 ng/ml nerve growth factor (NGF; Sigma).

The PC12 cells provided as described: above were treated with the inventive composition to a concentration of 10 μM, and after 24 hours, treated with the Aβ₁₋₄₂ protein to a concentration of 30 μM, and then cultured for 24 hours. In addition, a control group treated with only PBS, a group treated with only the inventive composition, and a group treated with only the amyloid beta-protein Aβ₁₋₄₂, were also provided for use in subsequent cell toxicity tests.

4) Cell Toxicity Test-1 (MTT Assay)

For MTT assay, 30 μM Aβ protein was added to the cultured cells and cultured under conditions of 5% CO₂ and 37° C. After 24 hours, a solution of MTT (3-(4,5-dimethylthiazol-2-yl)-2,5-diphenyltetrazolium bromide (Sigma Co.) was added to each well to a concentration of 0.5 mg/ml and then cultured for 4 hours and 30 minutes. A formazan precipitate formed by MTT reduction was dissolved in dimethyl sulfoxide (DMSO), and then, measured for the absorbance at 570 nm using an ELISA reader. The results of cell toxicity measurement for each sample were calculated as relative values, assuming that absorbance measured in the negative control group to which only solvent was added is 100%, and absorbance measured in the positive control group whose cells were completely broken by 0.9% Triton X-100 is 0%. In this case, the lower the MTT reduction, the lower the cell viability.

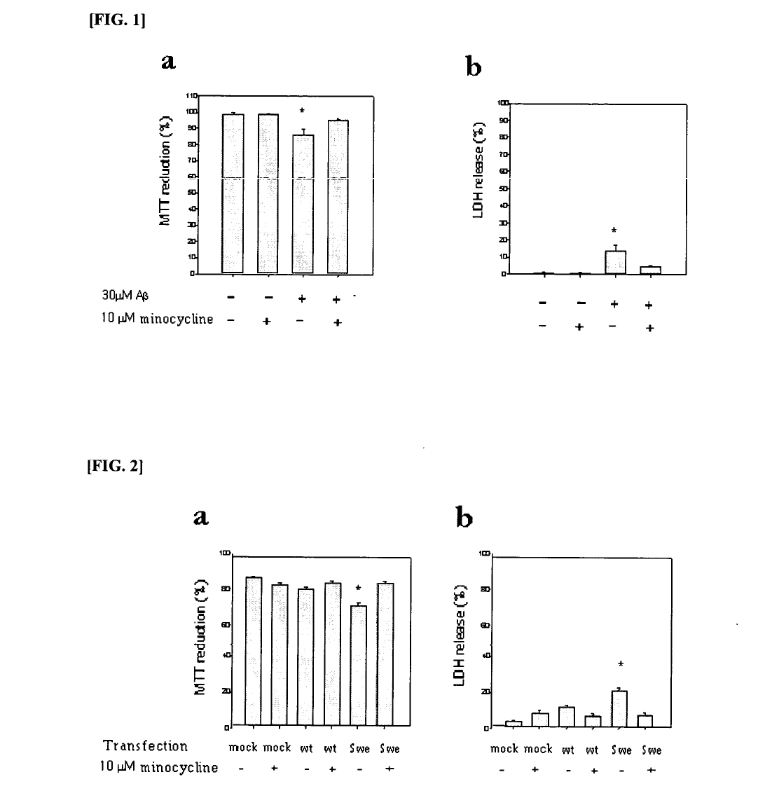

As described above, the MTT reduction was measured to obtain the cell viability of each group. The measured results are shown in FIG. 1a.

As shown in FIG. 1a, the results of the MTT assay showed that brain cell viability was significantly reduced by the extracellularly treated amyloid beta-protein Aβ₁₋₄₂ (the third bar graph indicated by the symbol * in FIG. 1a), but it was remarkably recovered in the case of pretreatment with the composition of the present invention (the fourth bar graph in FIG. 1a).

5) Cell Toxicity Test-2 (LDH Assay)

LDH assay, which is a method of measuring the amount of lactate dehydrogenase (LDH) released into medium with cellular membrane breakdown, was conducted using a CytoTox 96 assay kit (Promega). Peptides of a given concentration were added to the cultured cells, and then, cultured under conditions of 5% CO₂ and 37° C. for 24 hours. After each 50 ml of the culture broths were collected, 50 ml of a substrate mix consisting of NAD+ and tetrazolium salt was added to each well and allowed to react in a darkroom for 30 minutes. As a red color was expressed by formazan formed by LDH, the reaction was terminated by the addition of stop solution, and the absorbance at 490 nm was measured using an ELSA reader. LDH release for the respective groups was calculated as a relative value, assuming that LDH release obtained in cells, which were completely dissolved with 0.9% Triton X-100, is 100%. In this case, the higher the LDH release, the lower the cell viability.

As described above, the LDH release was measured to obtain the cell viability of each group. The measured results are shown in FIG. 1b.

As shown in FIG. 1b, the results of LDH assay showed that brain cell viability was significantly reduced by the extracellularly treated amyloid beta-protein Aβ₁₋₄₂ (the third bar graph indicated by the symbol * in FIG. 1b), but it was remarkably recovered in the case of pretreatment with the composition of the present invention (the fourth bar graph in FIG. 1b).

### 6) CONCLUSION

From the results of the MTT and LDH assay, it can be found that the composition of the present invention can effectively inhibit brain cell death induced by extracellularly treated amyloid beta-protein.

### EXAMPLE 2

### Effect of Inventive Composition on Brain Cell Death Induced by Intracelluarly Produced Amyloid Beta-Protein

The effect of the composition of the present invention on brain cell death induced by intracellularly produced amyloid beta-protein was examined as follows.

1) Production of Inventive Composition

The composition of the present invention was produced in the same manner as in the section 1) of Example 1.

2) Preparation of Amyloid Beta-Protein-Expressing Cells

For the preparation of amyloid beta-protein-expressing cells, recombinant vectors were constructed which can express the wild type (hereinafter, referred to as “WT”) and Swedish mutant (hereinafter, referred to as “swe”) amyloid precursor proteins. The Swedish mutant protein is known as being frequently found in the brain of patients showing early symptoms of dementia.

In order to obtain base sequences coding for genes of such proteins, primers to be used in PCR using amyloid precursor protein as a template were constructed as given in Table 1 below.

TABLE 1

| Target protein | Direction | Base sequence | SEQ ID NO |
|---|---|---|---|
| WT | Forward | 5′-AGTTTCCTCGGCAGCGGTAGGCGAGA-3′ | SEQ ID NO: 1 |
| | Reverse | 5′GTTCTGCATCTGCTCAAAGAACTTGTA-3′ | SEQ ID NO: 2 |
| Swe | Forward | AGTTTCCTCGGCAGCGGTAGGCGAGA-3′ | SEQ ID NO: 3 |
| | Reverse | GTTCTGCATCTGCTCAAAGAACTTGTA-3′ | SEQ ID NO: 4 |

After conducting PCR using the primers, the amplified DNAs thus obtained were subjected to agarose gel electrophoresis, and DNA band sizes were compared to confirm if the desired genes were correctly obtained.

Each of the PCR products was cloned into eukaryote-expressing vector pCB6 having a T7 promoter and capable of producing a myc fusion protein, thereby conducting recombinant plasmids.

Cells to be used in transduction using such recombinant plasmids were cultured in the same manner as in the section 3) of Example 1. After 24 hours, 2 μg of each of the recombinant plasmids produced as described above was added to 100 μl medium containing 3 μl transduction regent (Fugene 6, BM), and allowed to react at room temperature for 30 minutes. The solution was slowly added dropwise to the culture medium of the cells, and cultured under conditions of 5% CO₂ and 37° C. In addition, the following groups were also provided: A control group where treatment and also transduction were not conducted; a group which was treated with only the composition of the present invention; and a group where only transduction was conducted.

At 6 hours after transduction, the culture medium was replaced by new culture medium, and treated with the composition of the present invention to a concentration of 10 μM, and then cultured for 24-48 hours. Following this, the cells were collected for use in subsequent cell toxicity tests.

3) Cell Toxicity Test-I (MTT Assay)

MTT reduction was measured for the collected cell of each of the groups in the same manner as in the section 4) of Example 1. Cell viability obtained from the measured MTT reduction is shown in FIG. 2a.

As shown in FIG. 2a, cell viability was not particularly influenced by the intracellularly produced amyloid precursor protein as known in the prior art (the third bar graph in FIG. 2a), but significantly reduced by the intracellularly produced amyloid beta-protein swe (the fifth bar graph indicated by the symbol * in FIG. 2a). On the other hand, in the group which had been pretreated with minocycline before the swe protein was not formed intracellularly, cell viability was remarkably reduced (the sixth bar graph in FIG. 2a).

4) Cell Toxicity Test-2 (LDH Array)

The cell of each of the groups was measured for LDH release in the same manner as in the section 5) of Example 1. Cell viability obtained from the measured LDH release is shown in FIG. 2b.

As shown in FIG. 2b, cell viability was not particularly influenced by the intracellularly produced amyloid precursor protein as known in the prior art (the third bar graph in FIG. 2b), and was significantly reduced by the intracellularly produced amyloid beta-protein swe (the fifth bar graph indicated by the symbol * in FIG. 2b). On the other hand, in the group which had been pretreated with minocycline before the swe protein was not formed intracellularly, cell viability was remarkably reduced (the sixth graph bar in FIG. 2b).

5) Cell Toxicity Test-3 (Cyt C Assay)

The intracytoplasmic expression pattern of cytochrome C (Cyt C) having connection with cell toxicity was examined by Western blotting.

The cells cultured as described above were dissolved in solubilizing buffer, and precipitated in a centrifuge at 16,000×g for 10 minutes. The supernatant was collected and subjected to protein quantification.

For electrophoresis, the respective proteins having the same concentration (30-60 μg) were boiled in sample buffer together and then subjected to SDS-PAGE under denaturing condition. Upon this electrophoresis, a standard size marker was also used to confirm isolation of the beta-amyloid precursor protein and the swe protein by the size of detected protein bands.

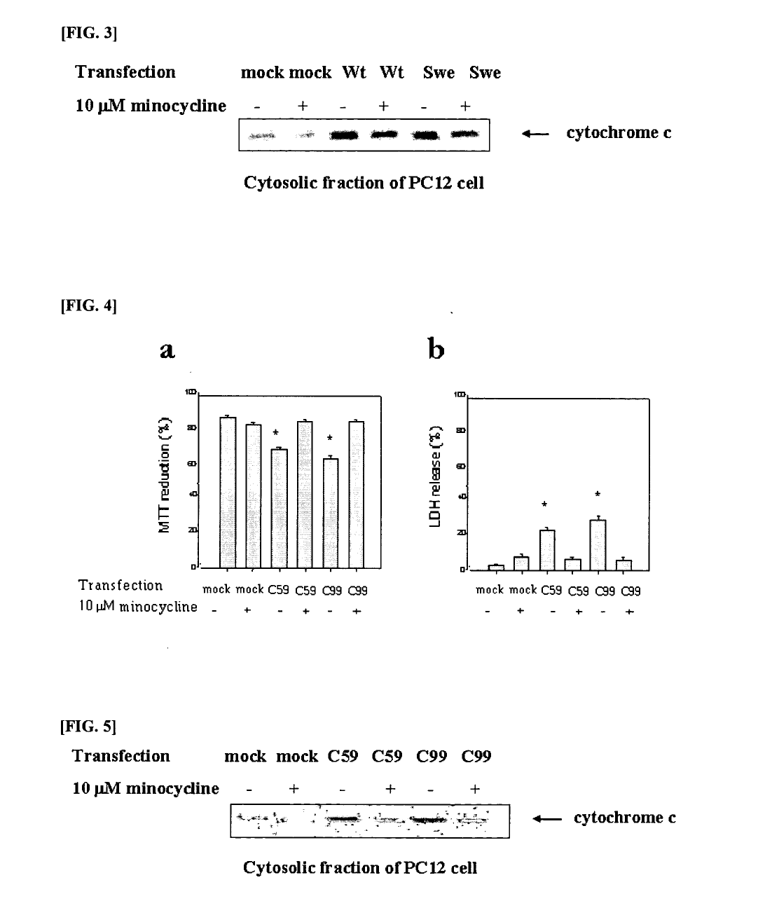

After electrophoresis, the proteins on gel were transferred to a membrane. The membrane was blocked with skim milk solution, washed three times with 0.05% Tween 20 TBS, and allowed to react with an antibody to the beta-amyloid precursor protein and the swe protein for one hour. After the first antibody reaction, it was reacted with an HRP-conjugated secondary antibody, and the expression of Cyt C protein was examined using an enhanced-chemiluminiscent detection kit (ECL). The examined results are shown in FIG. 3.

As shown in FIG. 3, in the control group, the release of Cyt C, a cell toxicity-associated protein, in the cytoplasmic layer, was increased by the intracellularly produced beta-amyloid precursor protein and the swe protein (the third and fifth in FIG. 3). On the other hand, in the group treated with the inventive composition, Cyt C release caused by such proteins was reduced (the fourth and sixth in FIG. 3).

### 6) CONCLUSION

From the results of the MTT, LDH and Cyt C assay, it can be found that the composition of the present invention can effectively inhibit brain cell death induced by intracellularly produced amyloid beta-protein.

### EXAMPLE 3

### Effect of Inventive Composition on Brain Cell Death Induced by Intracellularly Produced C-Terminal Protein

The effect of the composition of the present invention on brain cell death induced by intracellularly produced C-terminal protein was examined as follows.

1) Preparation of the Inventive Composition

the composition of the present invention was prepared in the same manner as the section 1) of Example 1.

2) Preparation of C-Terminal Protein

For the preparation of C-terminal protein-expressing cells, recombinant vectors were constructed which can express the C-terminal 59 amino acid fragment of amyloid precursor protein (hereinafter, referred to as “C59”), and the C-terminal 99 amino acid fragment of amyloid precursor protein (hereinafter, referred to as “C99”).

To obtain base sequences coding for genes of such proteins, primers to be used in PCR using amyloid precursor protein as a template were constructed as given in Table 2 below.

TABLE 2

| Target protein | Direction | Base sequence | SEQ ID NO |
|---|---|---|---|
| C59 | Forward | 5′-ATAGAGACAGTGATCGTCATCACCTTG-3′ | SEQ ID NO: 5 |
| | Reverse | 5′-GTTCTGCATCTGCTCAAAGAACTTGTA-3′ | SEQ ID NO: 6 |
| C99 | Forward | 5′-GATGCAGAATTCGGACATGACTGAGGA-3′ | SEQ ID NO: 7 |
| | Reverse | 5′-GTTCTGCATCTGCTCAAAGAACTTGTA-3′ | SEQ ID NO: 8 |

After conducting PCR using the primers, the amplified DNAs thus obtained were subjected to agarose gel electrophoresis, and DNA band sizes were compared to confirm if the desired genes were correctly obtained.

Each of the PCR products was cloned into eukaryote-expressing vector pEGFP-N1 having a T7 promoter and capable of producing a myc fusion protein, thereby constructing recombinant plasmids.

Then, transduction using such recombinant plasmids and cell collection were conducted in the same manner as the section 2) of Example 2.

3) Cell Toxicity Test-3 (MTT Assay)

The cell of each of the groups was measured for MTT reduction in the same manner as the section 4) of Example 1. Cell viability obtained from the measured MTT reduction is given in FIG. 4a.

As shown in FIG. 4a, brain cell viability was significantly reduced by the intracellularly produced C-terminal proteins, such as C59 and C99 (the third and fifth bar graphs each indicated by the symbol * in FIG. 4a), but this cell death effect was greatly reduced by pretreatment with minocycline (the fourth and sixth bar graphs in FIG. 4a).

4) Cell Toxicity Test-2 (LDH Assay)

The cell of each of the groups was measured for LDH release in the same manner as the section 5) of Example 1. Cell viability obtained from the measured MTT reduction is given in FIG. 4b.

As shown in FIG. 4b, brain cell viability was significantly reduced by the intracellularly produced C-terminal proteins, such as C59 and C99, (the third and fifth bar graphs each indicated by the symbol * in FIG. 4b), but this cell death effect was greatly reduced by pretreatment with minocycline (the fourth and sixth bar graphs in FIG. 4b).

5) Cell Toxicity Test-3 (Cyt C Assay)

The intracytoplasmic expression pattern of Cyt C having connection with cell toxicity was examined by Western blotting.

The cells cultured as described above were dissolved in solubilizing buffer, and precipitated in a centrifuge at 16,000×g for 10 minutes. The supernatant was collected and subjected to protein quantification.

For electrophoresis, the respective proteins having the same concentration (30-60 μg) were boiled in sample buffer together and then subjected to SDS-PAGE under denaturing condition. Upon this electrophoresis, a standard size marker was also used to confirm isolation of the beta-amyloid precursor protein and the swe protein by the size of detected protein bands.

After electrophoresis, the proteins on gel were transferred to a membrane. The membrane was blocked with skim milk solution, washed three times with 0.05% Tween 20 TBS, and allowed to react with an antibody to C59 and C99 proteins for one hour. After the first antibody reaction, it was reacted with an HRP-conjugated secondary antibody, and the expression of Cyt C protein was examined using an ECL kit. The examined results are shown in FIG. 5.

As shown in FIG. 5, in the control group, the release of cell toxicity-associated protein Cyt C into the cytoplasmic layer was increased by C59 and C99 proteins (the third and fifth in FIG. 5). On the other hand, in the group treated with the inventive composition, Cyt C release caused by such proteins was reduced (the fourth and sixth in FIG. 5).

### 6) CONCLUSION

From the results of the MTT, LDH and Cyt C assay, it can be found that the composition of the present invention can effectively inhibit brain cell death induced by intracellularly produced C-terminal protein.

### EXAMPLE 4

### Effect of Inventive Composition on Simple Memory Impairment Induced by C-Terminal Protein

1) Preparation of the Preparation of the Present Invention

Minocycline was dissolved in PBS buffer (pH 7.4) to a concentration of 5 mg/ml to prepare the composition of the present invention.

2) Preparation of C-Terminal Protein

As C-terminal protein to be used in dementia induction, the C-terminal 105 amino acid fragment of amyloid precursor protein (hereinafter, referred to as “CT105”) was prepared as followed. The desired genes were obtained by PCR using primers having the following base sequences.

| | |
|---|---|
| Forward: | |
| 5′-ATCTCTGAAGTGAAGATGGATGCAGAA-3′ | (SEQ ID NO: 9) |
| Reverse: | |
| 5′-GTTCTGCATCTGCTCAAAGAACTTGTA-3′ | (SEQ ID NO: 10) |

The genes described above were cloned into prokaryote-expressing vector pGEX4T-1 having a Trp promoter.

E. coli was transformed with the resulting recombinant plasmids (Hanahan). To confirm protein expression, a colony appearing on solid medium was sub-cultured overnight, and inoculated to M9 medium and then bulk-cultured. As the absorbance at 600 nm reached about 0.5-0.8, isopropylthiogalactoside (PTG) was added to a final concentration of 1 mM to induce protein expression. Protein was obtained from the cultured cell obtained as described above, and SDS-PAGE analysis was conducted according to the method of Lammeli (1970), to confirm expression of the recombinant protein.

The cells were precipitated from the cell culture medium, and suspended in STET buffer, and then treated with lysozyme for a given period of time. The cells were homogenized with a sonicator and then centrifuged at about 6,000 rpm to obtain insoluble pellets. Such pellets were washed with NTE buffer, and then, successive 2M-4M-8M urea extraction processes were conducted while setting a purification process depending on the degree of expression, thereby partially purifying proteins.

Non-fused recombinant proteins were purified using methods, such as gel filtration, ion exchange chromatography and electroelution. Fusion partners for the purified proteins were cut off to obtain pure recombinant proteins to be used in dementia induction.

3) Breeding of Test Animals

For use as dementia-induced animal models, male ICR mice (18-26 g body weight) were selected, and bred at room temperature (25° C.) under a 12 hr light/dark cycle over a test period.

Before a test for memory estimation, the composition of the present invention produced as described above was administered intra-abdominally at a minocycline dose of 10 mg/kg a time a day for one week. Also, groups which had not been administered with the inventive composition were bred under the same condition.

4) Estimation of Simple Memory Ability by Passive Avoidance Test

By passive avoidance test, mice to which the inventive composition had been administered or had not been administered was allowed to learn and then estimated for simple memory after dementia induction.

As a test device, an automated shuttle box (Model PACS-30, Columbus Instruments International Company) was used. The shuttle box was divided into two compartments of the same size (19 cm long×9 cm wide×10.875 cm high) by a partition door (3 cm long×2.625 cm wide). A bottom of the compartments was equipped in such a manner as to permit electric current passage, and the respective compartments were illuminated by a 20-W bulb. Thus, the mice housed in the box could run from a light compartment to a dark compartment via the partition door.

The shuttle box was placed in a room where a noise was kept below 60 dB and illumination became dark. One compartment of the shuttle box was electrically lighted and white rats were placed in the lighted compartment. As the white rats crossed the partition door into the other compartment, the partition door was automatically shut. The time required for the white rats to run from the lighted compartment to the unlighted compartment was taken as latency time, and was used as a measure of subsequent memory estimation. The process described above was repeated to make the white rats learn to run from the lighted compartment to the unlighted compartment within 20 seconds. After 24 hours, the white rats were placed in the lighted compartment again. As the white rats crossed into the unlighted compartment, the partition door was shut while an electric current of 1 mA was passed through the bottom for 3 seconds.

At 30 minutes after the learning process as described above, the CT105 protein produced in the section 2) of Example 4 was dissolved in 0.1M phosphate-buffered saline (PBS; pH 7.4), and administered into the cerebral ventricle of the test animals at a dose of 685 pmol/5 ml using a Hamilton syringe. In addition, the following groups were also provided: A control group to which any agent was not administered; and a comparison group which was administered with only the CT105 protein for dementia induction without pretreatment with minocycline.

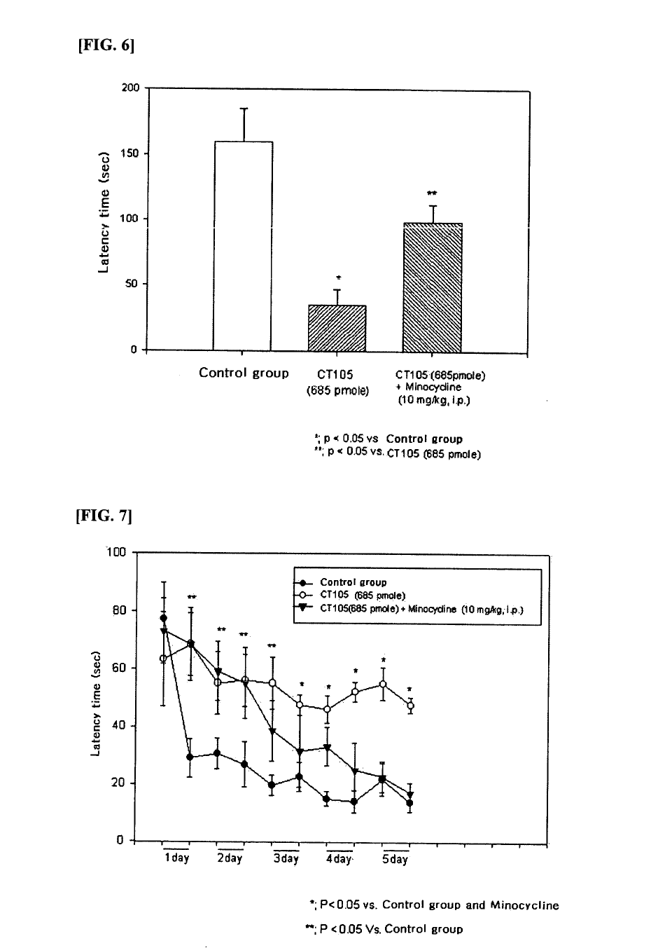

After one week, the mice of each group were placed in the shuttle box again, and the time required to run from the lighted compartment to the unlighted compartment was measured. A change in latency time shows memory impairment and recovery, and an increase latency time shows that memory was increased. The longest latency time was 5 minutes, and each of the tests was separately conducted three times or more, and statistical significance was verified by analysis of variance (ANOVA) or student t-test. The test results are shown in FIG. 6.

As shown in FIG. 6, latency time for the mice whose cerebrum had been injected with the CT105 protein was statistically significantly shorter than the control group (the second bar graph by the symbol * in FIG. 6). This is because dementia was induced by the deposition of the CT105 protein to impair simple memory. However, latency time for the group which had been administered with the CT105 protein after pretreatment with the inventive composition was significantly reduced as compared to the comparison group which had been administered with only the CT105 protein (the third bar graph indicated by the symbol ** in FIG. 6).

### 5) CONCLUSION

By the passive avoidance test as described above, it can be found that the composition of the present invention can prevent the impairment of simple memory induced by the C-terminal fragment of amyliod precursor protein.

### EXAMPLE 5

### Effect of Inventive Composition on Spatial Memory Impairment Induced by C-Terminal Protein

The effect of the inventive composition on spatial memory impairment induced by C-terminal protein was examined as follows.

1) Preparation of the Composition of the Present Invention

The composition of the present invention was prepared in the same manner as the section 1) of Example 4.

2) Preparation of C-Terminal Protein

The C-terminal protein to be used in dementia was prepared in the same manner as the section 2) of Example 4.

Animals to be used as dementia-induced animal models were bred and pretreated with minocycline in the same manner as the section 3) of Example 4. After pretreatment with minocycline, the animals were treated with the C-terminal protein in the same manner as the section 4) of Example 4. In addition, the following groups were also provided: A control group to which any agent was not administered; and a comparison group which was administered with only the C-terminal protein.

4) Estimation of Spatial Memory by Water Maze Test

By a water maze test, spatial memory after dementia induction was estimated for mice which had been administered or had not been administered with the composition of the present invention.

Water was charged into a cylindrical water tank with a diameter of 140 cm and a height of 45 cm, and then made opaque by the addition of milk. The inside of the water tank was randomly divided into four zones, and a safety plate was disposed in one zone of the four zones at about 1.5 cm below the water surface such that it was invisible.

If the mice of the respective groups are placed into the cylindrical water tank, starting from a location of the farthest side away from the safety plate, the mice will look out for and go on the safety plate with swimming. In this case, the time required for the mice to reach the safety plate from the starting location was taken as latency time. The longest observation time was 90 seconds, and if the mice fail to locate the safety plate within 90 seconds, they were put on the safety plate for 15 seconds. At 1 minute after the first test, the second test was conducted. Obtained data were analyzed with a video image motion analyzer (Ethovision, Noldus Information Technology).

The mice of each group were subjected to the water maze test two times in the morning and two times in the afternoon for five days. Each of the tests was separately conducted three times or more, and statistical significance was verified by analysis of variance or student t-test. The test results are shown in FIG. 7.

As shown in FIG. 7, latency time for the mice whose cerebral ventricle had been injected with the CT105 protein was statistically significantly shorter than the control group. This is because dementia was induced by the deposition of the CT105 protein to impair spatial memory. However, latency time for the group which had been administered with the CT105 protein after pretreatment with the inventive composition was significantly reduced as compared to the comparison group which had been administered with only the CT105 protein).

### 5) CONCLUSION

By the water maze test as described above, it can be found that the composition of the present invention can prevent the impairment of spatial memory induced by the C-terminal fragment of amyliod precursor protein.

### EXAMPLE 6

### Effect of Inventive Composition on Alzheimer's Disease Induced by Beta-Amyloid Protein

The effect of the composition of the present invention on Alzheimer's disease induced by beta-amyloid protein was examined as follows.

1) Preparation of the Composition of the Present Invention

Minocycline was dissolved in PBS buffer (pH 7.4) to a concentration of 10 mM to prepare the composition of the present invention.

2) Preparation of Amyloid Beta-Protein

As amyloid beta-protein to be used in the induction of Alzheimer's disease, an Aβ₁₋₄₂ protein manufactured by U.S. Peptide Inc. was purchased.

3) Construction of Test Animal Models

To induce Alzheimer's disease, beta-amyloid infusion models, in which an osmotic mini-pump (Alzet Co., USA) is placed into the brain of test animals and attached to a brain infusion kit (Alzet Co., USA), were constructed as follows.

Test animals were anesthetized with sodium pentobarbital (3 ml/kg), and the brain infusion kit was placed into the right brain ventricle (AP: −0.8 mm; ML: 1.2 mm; DV: −3.6 mm) of the test animal.

The Aβ₁₋₄₂ protein was dissolved in a mixed solution of sterilized saline containing 35% acetonitrile and 0.1% trifluoroacetic acid. The resulting beta-amyloid protein solution was charged into the osmotic mini-pump which was then attached to the brain infusion kit. Next, the protein solution was infused into the brain ventricle for one week.

The test animals were divided into a control group, a comparison group and a test group to be administered with the inventive composition, each group consisting of generally 10 animals. The control group was infused with PBS and the comparison group was infused with the beta-amyloid protein solution.

After one week, the composition of the present invention was administered intra-abdominally at a dose of 45 mg/kg/day for two weeks.

4) Estimation of Memory and Learning Ability by Water Maze Test

The animal models constructed in the above section 3) were subjected to Morris water maze test.

This water maze test was conducted according to Ethovision Program (Noldus Information Technology B.v., The Netherlands).

A water tank having a diameter of 1.4 m was filled with water and divided into four zones. Then, a platform was disposed at the center of one zone of the four zones. In four directions outside the water tank, several different markers allowing the test animals to recognize directions were disposed.

A learning trial where the test animals are placed into the water tank and allowed to look out for the platform was conducted two times a day for six days, and at the same time, the time required for the test animals to locate the platform was measured. At 48 hours after the last learning trial, the platform was removed from the water tank. In this state, a probe test was conducted, in which the test animals are placed into the water tank, and a time period where the test animals stay at the respective zones is measured.

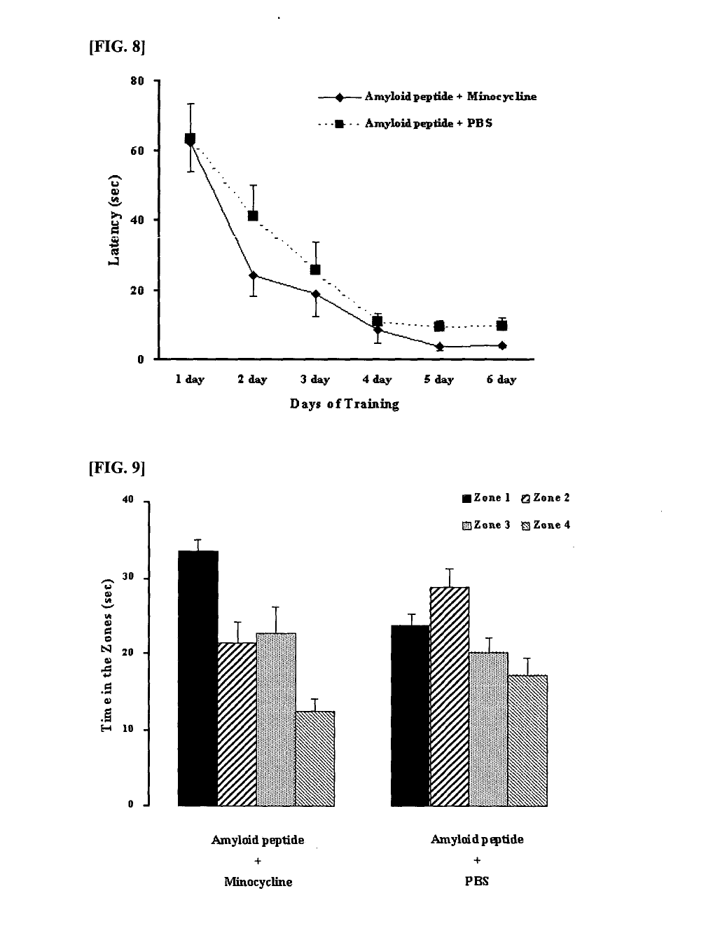

Each of the tests was separately conducted three times or more, and statistical significance was verified by analysis of variance or student t-test. The test results are shown in FIGS. 8 and 9.

As shown in FIG. 8, the test animal, which had been administered with the composition of the present invention, looked out for the platform far faster than the comparison group (Student's t-test, p<0.05).

As shown in FIG. 9, even after the platform was removed, the test group administered with the inventive composition stayed at zone 1 (where the platform was placed before removal) for a longer time than other zones 2, 3 and 4, and showed a statistically significant difference in the stayed time from the comparison group (one-way ANOVA, p<0.05).

### 5) CONCLUSION

From the test results, it can be found that the composition of the present invention can prevent the impairment of memory learning ability induced by Alzheimer's disease.

As described above, the composition of the present invention has an effect of inhibiting brain cell death induced by the toxicity of amyloid beta-protein or C-terminal protein.

Furthermore, the composition of the present invention has an effect of recovering the impairment of memory induced by amyloid beta-protein or C-terminal protein.

Accordingly, the composition of the present invention is useful for the prevention and treatment of various dementias, including Alzheimer's disease showing the above-mentioned symptoms as main symptoms.

1. A pharmaceutical composition for preventing and treating dementia, which contains minocycline as an active ingredient.

2. The pharmaceutical composition of claim 1, which inhibits brain cell toxicity.

3. The pharmaceutical composition of claim 2, which inhibits the brain cell toxicity of amyloid beta-protein.

4. The pharmaceutical composition of claim 2, which inhibits the brain cell toxicity of C-terminal protein.

5. The pharmaceutical composition of claim 1, which inhibits the impairment of learning and memory and cognitive function.

6. The pharmaceutical composition of claim 5, which inhibits the impairment of learning and memory and cognitive function induced by amyloid beta-protein.

7. The pharmaceutical composition of claim 5, which inhibits the impairment of learning and memory and cognitive function induced by C-terminal amyloid precursor protein.

8. The pharmaceutical composition of claim 1, wherein the dementia is Alzheimer's disease.

9. A pharmaceutical composition for preventing and treating the impairment of learning and memory and cognitive function which contains minocycline as an active ingredient.

10. The pharmaceutical composition of claim 9, which inhibits brain cell toxicity.

11. The pharmaceutical composition of claim 10, which inhibits the brain cell toxicity of amyloid beta-protein.

12. The pharmaceutical composition of claim 10, which inhibits the brain cell toxicity of C-terminal protein.

13. The pharmaceutical composition of claim 9, which inhibits the impairment of learning and memory and cognitive function induced by amyloid beta-protein.

14. The pharmaceutical composition of claim 9, which inhibits the impairment of learning and memory and cognitive function induced by C-terminal of amyloid precursor protein.

15. The pharmaceutical composition of claim 2, wherein the dementia is Alzheimer's disease.

16. The pharmaceutical composition of claim 3, wherein the dementia is Alzheimer's disease.

17. The pharmaceutical composition of claim 4, wherein the dementia is Alzheimer's disease.

18. The pharmaceutical composition of claim 5, wherein the dementia is Alzheimer's disease.

19. The pharmaceutical composition of claim 6, wherein the dementia is Alzheimer's disease.

20. The pharmaceutical composition of claim 7, wherein the dementia is Alzheimer's disease.


SEQUENCE LISTING (10 sequences)

SEQ ID NO: 1 - DNA, 26 bp, Artificial Sequence
  forward primer for amplifying wild type gene of amyloid precursor protein wild type
agtttcctcggcagcggtaggcgaga

SEQ ID NO: 2 - DNA, 27 bp, Artificial Sequence
  reverse primer for amplifying wild type gene of amyloid precursor protein wild type
gttctgcatctgctcaaagaacttgta

SEQ ID NO: 3 - DNA, 26 bp, Artificial Sequence
  forward primer for amplifying swedish mutant form of amyloid precursor protein wild type
agtttcctcggcagcggtaggcgaga

SEQ ID NO: 4 - DNA, 27 bp, Artificial Sequence
  reverse primer for amplifying swedish mutant form of amyloid precursor protein wild type
gttctgcatctgctcaaagaacttgta

SEQ ID NO: 5 - DNA, 27 bp, Artificial Sequence
  forward primer for amplifying the gene coding C- terminal 59 amino acids of amyloid precursor protein
atagcgacagtgatcgtcatcaccttg

SEQ ID NO: 6 - DNA, 27 bp, Artificial Sequence
  reverse primer for amplifying the gene coding C- terminal 59 amino acids of amyloid precursor protein
gttctgcatctgctcaaagaacttgta

SEQ ID NO: 7 - DNA, 27 bp, Artificial Sequence
  forward primer for amplifying the gene coding C- terminal 99 amino acids of amyloid precursor protein
gatgcagaattcggacatgactgagga

SEQ ID NO: 8 - DNA, 27 bp, Artificial Sequence
  reverse primer for amplifying the gene coding C- terminal 99 amino acids of amyloid precursor protein
gttctgcatctgctcaaagaacttgta

SEQ ID NO: 9 - DNA, 27 bp, Artificial Sequence
  forward primer for amplifying the gene coding C- terminal 105 amino acids of amyloid precursor protein
atctctgaagtgaagatggatgcagaa

SEQ ID NO: 10 - DNA, 27 bp, Artificial Sequence
  reverse primer for amplifying the gene coding C- terminal 105 amino acids of amyloid precursor protein
gttctgcatctgctcaaagaacttgta